(19) United States
(12) Patent Application Publication
(10) Pub. No.: US 2020/0227162 A1
(43) Pub. Date: Jul. 16, 2020

(54) HOSPITAL INFORMATION SYSTEM AND IMAGE DATA GENERATION METHOD

(71) Applicant: Canon Medical Systems Corporation, Otawara-shi (JP)

(72) Inventor: Hayato OKUMIYA, Nasushiobara (JP)

(73) Assignee: Canon Medical Systems Corporation, Otawara-shi (JP)

(21) Appl. No.: 16/833,958

(22) Filed: Mar. 30, 2020

Related U.S. Application Data
(63) Continuation of application No. PCT/JP2018/038682, filed on Oct. 17, 2018.

(30) Foreign Application Priority Data
Oct. 18, 2017 (JP) 2017-202106

(51) Int. Cl. G16H 40/20; G16H 20/10; G16H 10/60

(52) U.S. Cl. CPC G16H 40/20; G16H 10/60; G16H 20/10

(57) ABSTRACT

According to one embodiment, a hospital information system includes processing circuitry. The processing circuitry acquires information on an administration timing of a medicine to a patient and information on temporal changes of a medication effect of the medicine. The processing circuitry generates image data indicating the information on the administration timing and the information on temporal changes of the medication effect on a common time axis.

### CROSS-REFERENCE TO RELATED APPLICATIONS

This application is a Continuation Application of PCT Application No. PCT/JP2018/038682, filed Oct. 17, 2018 and based upon and claiming the benefit of priority from prior Japanese Patent Application No. 2017-202106, filed Oct. 18, 2017, the entire contents of all of which are incorporated herein by reference.

### FIELD

Embodiments described herein relate to a hospital information system and an image data generation method.

### BACKGROUND

On a conventional display for displaying the progress of medical care to a patient, a timing at which a medicine is to be administered is indicated by, for example, a dot, based on medicine administration information instructed by a doctor. On such a display, a period of time during which the medicine is to be administered is indicated by, for example, a band, based on the medicine administration information. Healthcare professionals such as doctors, nurses, pharmacists, etc. perceive the actual timing of administration of a medicine, which is directly related to their services in hospitals, based on the dots and bands displayed. Examples of healthcare professionals' services include handing medicines to patients, actually administering medicines, and checking the progress after the administration of the medicines.

In addition to the medicine administration timing and the medicine administration period, the display further displays, for example, values of examination results, vital signs of the patient, etc. in parallel. The vital signs include, for example, body temperature, pulse rate, blood pressure, respiratory rate, etc. Based on the contents displayed on the display, a doctor evaluates a relationship between the administration of a medicine as a medical care intervention and a response to the intervention.

Technically, the period of time over which a medicine exerts stable effects varies depending on the type of the medicine to be administered, the mode of administration to the patient into which the medicine is to be administered, the patient's condition, etc. Accordingly, the relationship between a medical care intervention and a response to the intervention might not be accurately evaluated based only on the medicine administration timing and the medicine administration period displayed on the display.

The problem to be solved by the present embodiments is to enable more precise evaluation of the relationship between a medical care intervention and a response to the intervention.

### BRIEF DESCRIPTION OF THE DRAWINGS

FIG. 1 is a block diagram illustrating an environment in which a hospital information system according to an embodiment is employed.

FIG. 2 is a block diagram showing a functional configuration of an electronic medical record server shown in FIG. 1.

FIG. 3 is a block diagram showing a functional configuration of a diagnostic server shown in FIG. 1.

FIG. 4 is a flowchart illustrating an operation of processing circuitry when the electronic medical record server shown in FIG. 2 acquires medication effect information.

FIG. 5 is a flowchart illustrating an operation of processing circuitry when the electronic medical record server shown in FIG. 2 generates display image data indicating temporal changes of a value representing the medication effect in a line graph.

FIG. 6 is a diagram illustrating a display image displayed on a display device included in the input/output apparatus shown in FIG. 1.

FIG. 7 is a flowchart illustrating an operation of the processing circuitry when the electronic medical record server shown in FIG. 2 generates display image data indicating temporal changes of the value representing the medication effect as variations in color.

FIG. 8 is a diagram illustrating a display image displayed on a display device provided by the input/output apparatus shown in FIG. 1.

FIG. 9 is a flowchart illustrating an operation of the processing circuitry when the electronic medical record server shown in FIG. 2 generates display image data indicating temporal changes of the value representing the medication effect by color transmittance.

FIG. 10 is a diagram showing a display image displayed on a display device provided by the input/output apparatus shown in FIG. 1.

FIG. 11 is a flowchart illustrating an operation of the processing circuitry when the electronic medical record server according to the modification analyzes various types of medical care information, and acquires the results of the analysis as medication effect information.

FIG. 12 is a diagram illustrating a first display example of a display image displayed on a display device included in an input/output apparatus of a diagnostic system according to another embodiment.

FIG. 13 is a diagram illustrating a second display example of a display image displayed on a display device included in an input/output apparatus of a diagnostic system according to another embodiment.

### DETAILED DESCRIPTION

In general, according to one embodiment, a hospital information system includes processing circuitry. The processing circuitry acquires information on an administration timing of a medicine to a patient and information on temporal changes of a medication effect of the medicine. The processing circuitry generates image data indicating the information on the administration timing and the information on temporal changes of the medication effect on a common time axis.

Hereinafter, embodiments will be described with reference to the accompanying drawings.

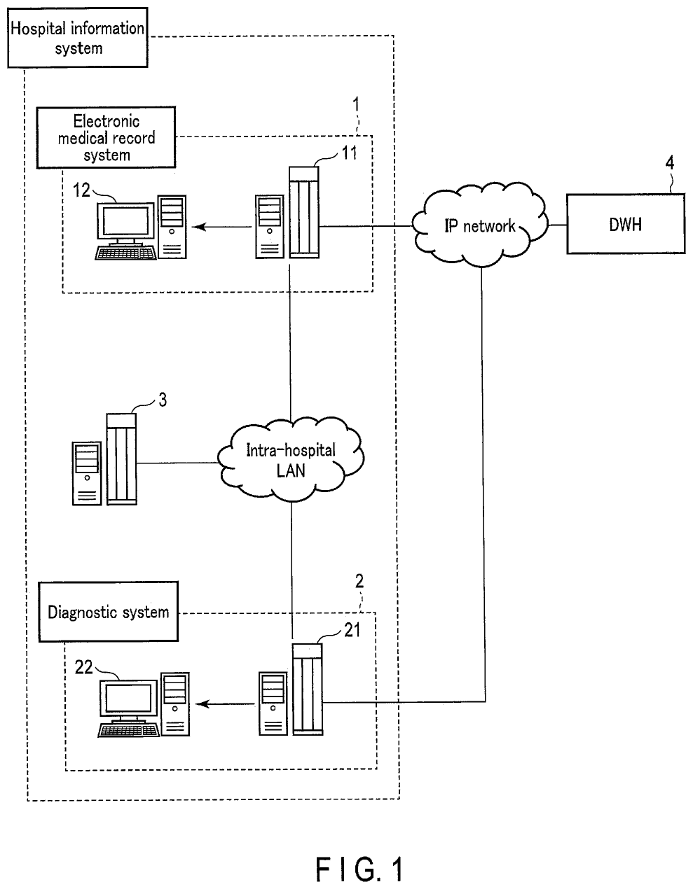

FIG. 1 is a block diagram illustrating an example of an environment in which a hospital information system according to the present embodiment is employed. The hospital information system shown in FIG. 1 includes an electronic medical record system 1, a diagnostic system 2, and a package insert information management server 3. In the present embodiment, let us assume that the electronic medical record system 1, the diagnostic system 2, and the package insert information management server 3 are, for example, connected to an intra-hospital network such as a local area network (LAN) to enable communications therebetween.

As shown in FIG. 1, the hospital information system is connected to, for example, a data warehouse (DWH) 4 via a security-ensured communication network. Examples of the security-ensured communication network include an inter-hospital network constructed by a dedicated line, a virtual private network (VPN), etc. The data warehouse 4 may be included in the hospital information system shown in FIG. 1. The hospital information system shown in FIG. 1 may be connected to, for example, a hospital information system of another hospital via a security-ensured communication network.

In FIG. 1, the electronic medical record system 1 is a system that manages electronic medical records (EMRs). Electronic medical records are medical care records created by doctors on a patient-by-patient basis. Information recorded in the electronic medical records includes patient information on patients and medical care information that is generated when medical care is provided to patients.

The medical care information is managed on, for example, a patient-by-patient basis. The patient information contains information on the patient's race, gender, age group, history of illness, name of the disease the patient is suffering from, drugs concurrently used, contraindications, allergies, etc. The medical care information contains values representing vital signs such as the body temperature, the pulse rate, the blood pressure, and the respiratory rate of each patient. The medical care information further contains actual records of administration of medicines.

The actual records of administration of medicines include the patient's responses to the past administration of pharmaceutical medicines (hereinafter simply referred to as “medicines”), which is a medical care intervention. The patient's response includes values summed up over a predetermined period of time, for every predetermined time interval since the start of administration of medicines. The values specifically representing the patient's response include, for example, the patient's examination value after administration of medicines, a changing situation of values representing the vital signs after the administration of the medicines with respect to expected values, the rate of change of values representing the vital signs after the administration of medicines, etc. The actual records of administration of medicines include types of medicines and modes of administration. The types of medicines include, for example, internal drugs, external drugs, and injection drugs. Examples of the mode of administration of medicines include the dosage, the procedure of administration, and the regimen of administration.

In the present embodiment, let us assume that the electronic medical records managed by the electronic medical record system 1 have preliminarily been analyzed by a predetermined data mining technique such as machine learning and statistical analysis. The analysis is performed by, for example, taking the patient information and the medical care information contained in the electronic medical records managed by the electronic medical record system 1 as input. The information output as a result of the analysis, which represents the patient's inherent tendency, is stored in the electronic medical record system 1 as first analyzed information.

The electronic medical record system 1 includes an electronic medical record server 11 and an input/output apparatus 12. The electronic medical record server 11 is a server that generates, based on the medication effect information, display image data for displaying the progress of medical care. In the present embodiment, the medication effect information is, for example, information in numerical form indicating the degree of effects to be exerted on the patient by the behavior of the medicine administered. The medication effect information contains, for example, the value of a blood concentration of an active ingredient in a predetermined medicine observed as a result of dissolution of the active ingredient into the patient's blood. The electronic medical record server 11 outputs the generated display image data to, for example, an input/output apparatus 12.

The input/output apparatus 12 is an apparatus for healthcare professionals such as doctors, nurses, and pharmacists to, for example, check and input the progress of medical care. The input/output apparatus 12 is constructed by, for example, a tablet computer, a personal computer, etc. The input/output apparatus 12 includes, for example, processing circuitry, an input interface, an output interface, and a communication interface.

The processing circuitry of the input/output apparatus 12 is a processor that serves as the nerve center of the input/output apparatus 12.

The input interface of the input/output apparatus 12 is constructed by, for example, a mouse, a keyboard, a touchpad to which an instruction is input via a touch on its operation surface, etc. The input interface receives, for example, a display instruction from an operator. The input interface converts the display instruction from the operator into an electrical signal, and outputs the electrical signal to the processing circuitry.

The output interface of the input/output apparatus 12 includes, for example, display interface circuitry and a display device. Examples of the display device that may be suitably employed include a CRT display, a liquid crystal display, an organic EL display, an LED display, a plasma display, and any other display known in the present technical field. The display interface circuitry converts data indicating a target to be displayed into a video signal. The display device displays a video signal converted by the display interface circuitry. The output interface may include a printing device. The printing device is, for example, a printer, and prints image data indicating a target to be printed on a predetermined sheet of paper. The output interface is not necessarily equipped with physical output components such as the display device and the printing device. Examples of the output interface include circuitry that transmits image data to an external output device provided separately from the input/output apparatus 12. The output interface of the input/output apparatus 12 displays an image based on the display image data which is output from the electronic medical record server 11.

A communication interface of the input/output apparatus 12 performs data communications with the electronic medical record server 11 connected thereto via an intra-hospital network. For example, the communication interface decodes the display image data transmitted from the electronic medical record server 11 according to a preset system, and outputs the decoded display image data to the processing circuitry.

In FIG. 1, the diagnostic system 2 is a system other than the electronic medical record system 1. Examples of the diagnostic system 2 include a picture archiving and communication system (PACS), a Vendor Neutral Archive (VNA) system, etc. The VNA system is a system that collectively manages diverse medical care information managed by systems of multiple clinical departments, such as a radiological information system (RIS) relating to radiographic examination, and an examination information system relating to physical examination. The diagnostic system 2 may be individually provided as a medical image management system or a clinical department system. The diagnostic system 2 may be either designed by a different vendor from the electronic medical record system 1 or by the same vendor as the electronic medical record system 1.

The diagnostic system 2 includes a diagnostic server 21 and an input/output apparatus 22. The diagnostic server 21 is a server that generates, based on the medication effect information, display image data for displaying the progress of medical care. The diagnostic server 21 outputs generated display image data to, for example, an input/output apparatus 22.

The input/output apparatus 22 is an apparatus for healthcare professionals such as doctors, nurses, and pharmacists to, for example, check and input the progress of medical care. The input/output apparatus 22 is constructed by, for example, a tablet computer, a personal computer, etc. The input/output apparatus 22 includes, for example, processing circuitry, an input interface, an output interface, and a communication interface. The input/output apparatus 22 displays an image based on the display image data which is output from the diagnostic server 21.

The package insert information management server 3 is a server that manages package insert information based on the description of package inserts included in pharmaceuticals. The package insert information contains, for example, information on the appearance of the medicine and an explanation of uses for which the medicine has been approved. The package insert information contains, for example, information explaining how the ingredients contained in the medicine functions in the patient's body into which the medicine has been administered, and how they are metabolized.

Specifically, the package insert information contains, for example, information on in vivo kinetics (pharmacokinetics). The information on in vivo kinetics contains information that is based on the biological half-life theory, which is information standardized for each pharmaceutical. The information standardized for each pharmaceutical contains information indicating, when a predetermined dose of a medicine is administered to a predetermined group of patients with a predetermined disease, an average value of temporal changes of the blood concentration of the active ingredient contained in the medicine administered to each patient in a predetermined mode. In other words, the information standardized for each pharmaceutical medicine contains information indicating the relationship between the blood concentration in a patient into which a pharmaceutical medicine has been administered, and the time elapsed since the administration of the medicine.

The data warehouse 4 is a database that collectively accumulates information generated in, for example, multiple medical-related and care-related institutions, namely, what is known as “medical care big data”. The data warehouse 4 stores, for example, patient information, medical care information, etc. generated in multiple medical-related and care-related institutions as medical care big data. Unlike the patient information contained in the electronic medical records, the patient information contained in the medical care big data does not contain personally identifiable information, such as name and address. That is, personally identifiable information is deleted from the patient information. In the present embodiment, let us assume that the patient information and medical care information accumulated in the data warehouse 4 are preliminarily analyzed by a predetermined data mining technique. The analysis is performed by, for example, taking the patient information and medical care information accumulated in the data warehouse 4 as input. The information output as a result of the analysis is stored in the data warehouse 4 as second analyzed information.

The electronic medical record server 11 included in the electronic medical record system 1 according to the present embodiment will be described in detail below.

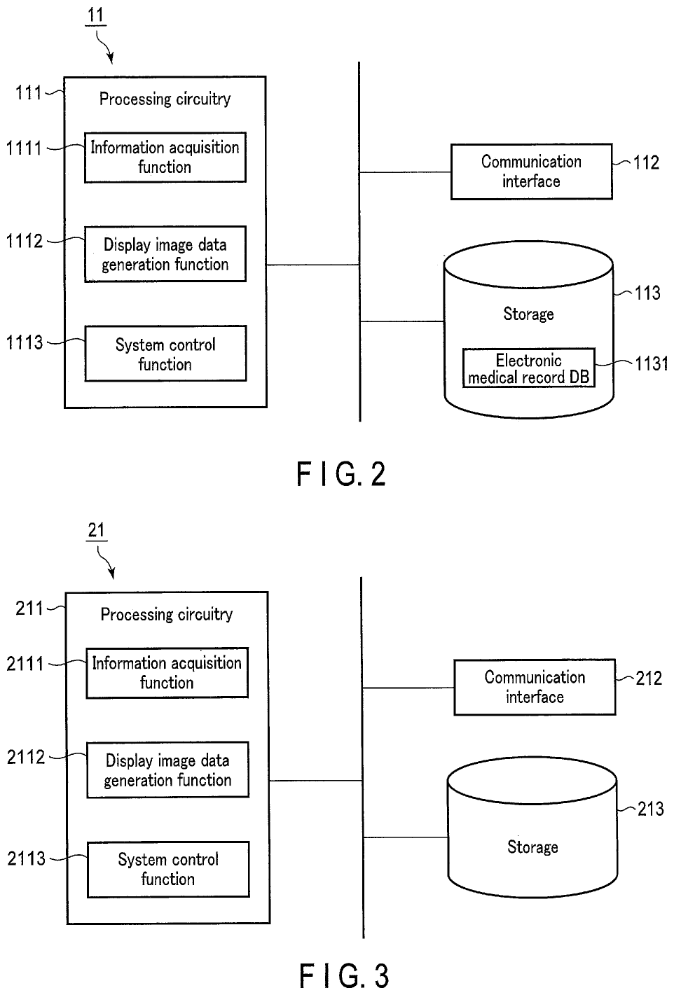

FIG. 2 is a block diagram showing an example of a functional configuration of the electronic medical record server 11 shown in FIG. 1. The electronic medical record server 11 shown in FIG. 2 includes processing circuitry 111, a communication interface 112, and storage 113. The processing circuitry 111, the communication interface 112, and the storage 113 are, for example, connected to each other via a bus. The electronic medical record server 11 may include an input interface, an output interface, etc.

The processing circuitry 111 is a processor that serves as the nerve center of the electronic medical record server 11. The processing circuitry 111 executes a processing program stored in the storage 113, thereby implementing the function corresponding to the executed program.

The communication interface 112 performs data communications with the input/output apparatus 12, the diagnostic system 2, and the package insert information management server 3, which are connected thereto via the intra-hospital network. For the communications with the input/output apparatus 12, the diagnostic system 2, and the package insert information management server 3, any standard may be used, such as Health Level Seven (HL7) and/or Digital Imaging and Communications in Medicine (DICOM). The communication interface 112 performs data communications with the data warehouse 4 connected thereto via a security-ensured communication network. For the communications with the data warehouse 4, any standard may be used, such as Internet Protocol (IP).

The storage 113 is a storage device that stores various types of information, such as a hard disk drive (HDD), a solid state drive (SSD), and an integrated-circuit memory device. The storage 113 may also be, for example, a drive that reads and writes various types of information on a portable storage medium, such as a CD-ROM drive, a DVD drive, a flash memory, etc. The storage 113 stores, for example, the processing program according to the present embodiment.

In the storage 113, an electronic medical record database (DB) 1131 is constructed. The electronic medical record database 1131 is managed by execution of the programs stored in the storage 113 by the processing circuitry 111.

The electronic medical record database 1131 is a database that stores the medical care information as electronic medical records. The electronic medical record database 1131 stores the medical care information as electronic medical records on, for example, a patient-by-patient basis or a medicine-by-medicine basis.

The processing circuitry 111 is, for example, a processor that serves as the nerve center of the electronic medical record server 11. The processing circuitry 111 executes an operation program stored in the storage 113, thereby implementing a function corresponding to the operation program. Specifically, the processing circuitry 111 is equipped with an information acquisition function 1111, a display image data generation function 1112, and a system control function 1113.

The information acquisition function 1111 is a function of acquiring information for generating medication effect information. The processing circuitry 111 periodically implements, for example, the information acquisition function 1111 at the timing when a preset batch process, etc. is performed. The processing circuitry 111 may implement the information acquisition function 1111 upon, for example, receiving a predetermined information acquisition instruction from the input/output apparatus 12 via the communication interface 112. When the information acquisition function 1111 is implemented, the processing circuitry 111 acquires at least one of the package insert information stored in the package insert information management server 3, the first analyzed information stored in the electronic medical record database 1131, and the second analyzed information stored in the data warehouse 4. Which of the package insert information, the first analyzed information, and the second analyzed information is to be acquired is, for example, preset as an option. The processing circuitry 111 stores the acquired package insert information, the first analyzed information, and the second analyzed information in the storage 113. Of the package insert information, the first analyzed information, and the second analyzed information, if two or more items of information are acquired, the processing circuitry 111 merges the acquired items of information, and stores the merged items of information in the storage 113.

The display image data generation function 1112 is a function of generating display image data for displaying the progress of medical care. The processing circuitry 111 executes the display image data generation function 1112. When the display image data generation function 1112 is executed, the processing circuitry 111 receives a display instruction to display the progress of the medical care via the communication interface 112 from, for example, the input/output apparatus 12. The display instruction contains, for example, information identifying the patient, the medicine, the display period, etc. for which healthcare professionals, etc. wish to look at the display.

Upon receiving the display instruction, the processing circuitry 111 reads information from the storage 113 based on the information on the patient, the medicine, and the display period contained in the display instruction. Based on the read information, the processing circuitry 111 calculates medication effect information, which indicates, in numerical form, the degree of effects to be exerted on the patient by the behavior of the medicine administered. For example, the processing circuitry 111 calculates, based on the merged items of information, an estimated blood concentration of an ingredient of a predetermined medicine in the patient as the medication effect information.

Based on the information on the patient and the display period contained in the display instruction, the processing circuitry 111 reads values representing the vital signs contained in the medical care information from the electronic medical record database 1131. Thereafter, the processing circuitry 111 generates display image data, in which the calculated medication effect information and the read values representing the vital signs are temporally associated. Thereby, display image data in which the calculated medication effect information and the values representing the vital signs are indicated on a common time axis during the display period included in the display instruction is generated.

The system control function 1113 is a function of controlling basic operations of the electronic medical record server 11, such as an output. When the system control function 1113 is executed, the processing circuitry 111 transmits the display image data generated by the display image data generation function 1112 to the input/output apparatus 12 via, for example, the communication interface 112.

The information acquisition function 1111, the display image data generation function 1112, and the system control function 1113 may be incorporated as control programs; alternatively, a dedicated hardware circuit capable of implementing each function may be incorporated in the processing circuitry 111 itself.

Hereinafter, the diagnostic server 21 included in the diagnostic system 2 according to the present embodiment will be described in detail.

FIG. 3 is a block diagram showing an example of a functional configuration of the diagnostic server 21 shown in FIG. 1. The diagnostic server 21 shown in FIG. 3 includes processing circuitry 211, a communication interface 212, and storage 213. The processing circuitry 211, the communication interface 212, and the storage 213 are, for example, connected to one another via a bus to enable communications therebetween. The diagnostic server 21 may include an input interface, an output interface, etc. included in the input/output apparatus 22.

The processing circuitry 211 is a processor that serves as the nerve center of the diagnostic server 21. The processing circuitry 211 executes a processing program stored in the storage 213, etc., thereby implementing a function corresponding to the program. Specifically, the processing circuitry 211 is equipped with an information acquisition function 2111, a display image data generation function 2112, and a system control function 2113. The functions equipped in the information acquisition function 2111, the display image data generation function 2112, and the system control function 2113 are the same as the functions equipped in the information acquisition function 1111, the display image data generation function 1112, and the system control function 1113, respectively, of the processing circuitry 111 of the electronic medical record server 11.

The communication interface 212 performs data communications with the input/output apparatus 22, the electronic medical record system 1, and the package insert information management server 3, which are connected thereto via an intra-hospital network. For the communications with the input/output apparatus 22, the electronic medical record system 1, and the package insert information management server 3, any standard may be used, such as HL7 and/or DICOM. The communication interface 212 performs data communications with the data warehouse 4 connected thereto via a security-ensured communication network. For the communications with the data warehouse 4, any standard may be used, such as IP.

The storage 213 is a storage device that stores various types of information, such as an HDD, an SSD, and an integrated-circuit memory device. The storage 213 may also be, for example, a drive that reads and writes various types of information on a portable storage medium, such as a CD-ROM drive, a DVD drive, a flash memory, etc. The storage 213 stores, for example, a processing program according to the present embodiment.

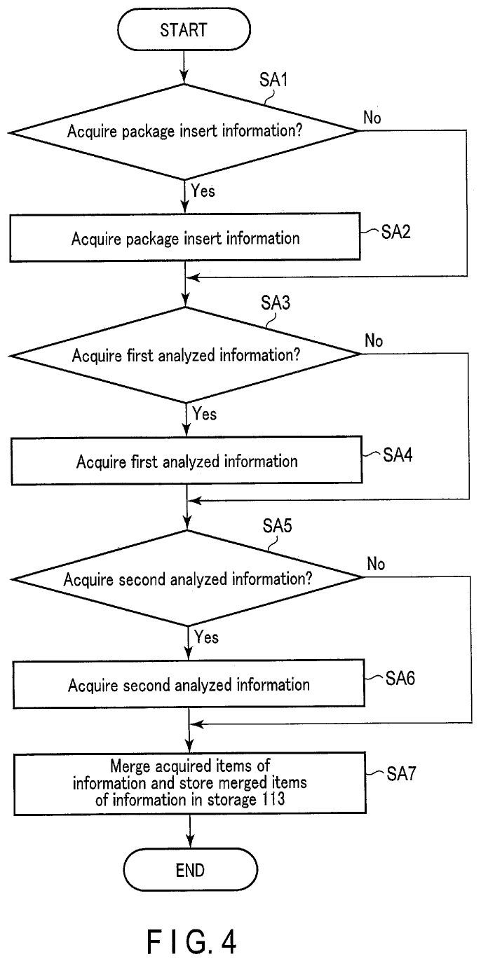

Next, various operations of the electronic medical record server 11 according to the present embodiment will be described with reference to the accompanying drawings. First, an operation through which the electronic medical record server 11 acquires information for generating medication effect information will be described. FIG. 4 is a flowchart illustrating an operation of the processing circuitry 111 when the electronic medical record server 11 shown in FIG. 2 acquires necessary information. In the description that follows, let us assume that options to acquire package insert information and first analyzed information are set. The information on the options is stored in, for example, the storage 113.

The processing circuitry 111 implements the information acquisition function 1111 when, for example, a preset batch process is started. Through the implementation of the information acquisition function 1111, the processing circuitry 111 refers to the information on the options stored in the storage 113, and determines whether the package insert information is an object to be acquired (step SA1). Upon determining that the package insert information is the object to be acquired (Yes in step SA1), the processing circuitry 111 acquires package insert information from the package insert information management server 3 via the communication interface 112 (step SA2).

Next, the processing circuitry 111 refers to the information on the options stored in the storage 113, and determines whether or not the first analyzed information is an object to be acquired (step SA3). Upon determining that the first analyzed information is the object to be acquired (Yes in step SA3), the processing circuitry 111 acquires first analyzed information from the electronic medical record database 1131 (step SA4).

Next, the processing circuitry 111 refers to information on the options stored in the storage 113, and determines whether or not the second analyzed information is an object to be acquired (step SA5). It is to be noted that the second analyzed information is not adopted as a target to be acquired in the preset options described herein. Upon determining that the second analyzed information is not the object to be acquired (No in step SA3), the processing circuitry 111 does not acquire second analyzed information from the data warehouse 4.

Of the package insert information, the first analyzed information, and the second analyzed information, the processing circuitry 111 merges the package insert information and the first analyzed information that have actually been acquired, and stores the merged items of information in the storage 113 (step SA7). Examples of the merging method include calculating the average value, the central value, the maximum value, the minimum value, etc. of the values contained in each item of information. Examples of the merging method include combining complementary items of information. By merging the acquired items of information, it is possible to improve the precision in the medication effect information generated based on the merged items of information.

Next, an operation through which the electronic medical record server 11 according to the present embodiment generates display image data, which indicates temporal changes of the value representing the medication effect in a predetermined display format, will be described.

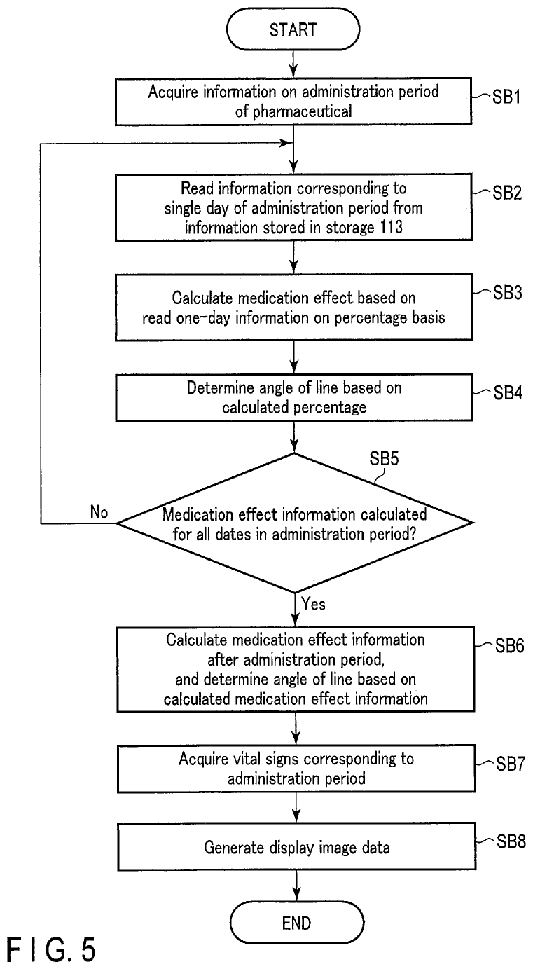

First, a case will be described where temporal changes of the value representing the medication effect are indicated in a line graph in a display image. FIG. 5 is a flowchart illustrating an example of an operation of the processing circuitry 111 when the electronic medical record server 11 shown in FIG. 2 generates display image data indicating temporal changes of the value representing the medication effect in a line graph.

In the illustration of FIG. 5, let us assume that doctors, nurses, etc. input a display instruction during, for example, a ward round for a regular temperature check, etc. of inpatients. In the illustration of FIG. 5, the display instruction contains information that identifies the names of medicines such as “AAA Tablet” and “BBB Tablet”. The display instruction also contains information that identifies the display period, “Feb. 23, 2017 (Thu) to Mar. 6, 2017 (Mon)”, taking into account the monitoring of the progress after administration.

In the illustration of FIG. 5, let us assume that the electronic medical record database 1131 stores first analyzed information obtained by analyzing medical care information on the patient to be the target of the display instruction at least during the period of Feb. 23, 2017 (Thu) to Mar. 6, 2017 (Mon). The first analyzed information contains, for example, “Feb. 23, 2017 (Thu) to Feb. 28, 2017 (Tue)” as the information indicating the period of administration of “AAA Tablet”, and “Take one tablet internally three times a day, e.g., after every meal in the morning, at noon, and in the evening” as the mode of administration of “AAA Tablet”. The first analyzed information also contains, for example, “Feb. 23, 2017 (Thu) to Feb. 24, 2017 (Fri)” as the information indicating the period of administration of “BBB Tablet”, and “Take one tablet internally once a day, e.g., after morning meal” as the mode of administration of “BBB Tablet”.

In the description of FIG. 5, let us assume that the data warehouse 4 stores second analyzed information obtained by analyzing the accumulated medical care information on the medicines “AAA Tablet” and “BBB Tablet”, for which the display instruction is to be made.

The processing circuitry 111 executes the display image data generation function 1112, and receives a display instruction to display the progress of medical care from, for example, the input/output apparatus 12 via the communication interface 112. The display instruction identifies the patient, the medicine, and the display period. Based on the medicine identified by the display instruction, the processing circuitry 111 acquires information on the period of administration and the mode of administration of the medicine from the electronic medical record database 1131 (step SB1).

Regarding the patient and the medicine identified by the display instruction, the processing circuitry 111 reads, from the information stored in the storage 113 in step SA7, information corresponding to a single day of the period of administration on which the information has been acquired (step SB2).

For example, the processing circuitry 111 reads one-day information corresponding to Feb. 23, 2017 (Thu) from information stored in the storage 113 and obtained by merging the package insert information and the first analyzed information. When the information obtained by merging the package insert information, the first analyzed information, and the second analyzed information is stored in the storage 113, the one-day information corresponding to Feb. 23, 2017 (Thu) is read from the merged items of information. The read one-day information contains, for example, a group of values representing the vital signs of an identified patient acquired at a given time interval during the period from 0 o'clock to 24 o'clock of Feb. 23, 2017 (Thu). The read one-day information contains, for example, information on the actual records of administration of the medicines “AAA Tablet” and “BBB Tablet” to an identified patient during the period from 0 o'clock to 24 o'clock of Feb. 23, 2017 (Thu). The read one-day information contains, for example, package insert information on each of the medicines “AAA Tablet” and “BBB Tablet”.

The processing circuitry 111 calculates medication effect information of Feb. 23, 2017 (Thu) based on the read one-day information for each patient and for each medicine identified by the display instruction (step SB3). In the present embodiment, the medication effect information is represented by, for example, the value of an estimated blood concentration of an ingredient of the identified medicine in the identified patient. For example, the processing circuitry 111 calculates, as medication effect information, blood concentration values on a percentage basis, assuming that the value of the blood concentration at which the ingredient of the identified medicine is considered to be sufficiently absorbed into the patient is 100.

Specifically, the processing circuitry 111 calculates, based on the read information of Feb. 23, 2017 (Thu), percentage values at a given time interval during the period from 0 o'clock to 24 o'clock of Feb. 23, 2017 (Thu), in relation to the blood concentration at which the medicine “AAA Tablet” has been sufficiently absorbed into the patient. Also, the processing circuitry 111 calculates, based on the read information of Feb. 23, 2017 (Thu), percentage values at a given time interval during the period from 0 o'clock to 24 o'clock of Feb. 23, 2017 (Thu), in relation to the blood concentration at which the medicine “BBB Tablet” has been sufficiently absorbed into the patient. The calculated medication effect information is, for example, a group of values calculated at a given time interval during the period from 0 o'clock to 24 o'clock of Feb. 23, 2017 (Thu).

The processing circuitry 111 determines an angle of a line to be displayed on the screen based on the calculated medication effect information, for each patient and medicine identified by the display instruction (step SB4). Specifically, the processing circuitry 111 determines, based on, for example, the medication effect information on each of the medicines “AAA Tablet” and “BBB Tablet”, calculated during the period from 0 o'clock to 24 o'clock of Feb. 23, 2017 (Thu), an angle of a line drawn from the value (0) at the time of the first medication to the value at the final point, with respect to the ruled line.

Subsequently, the processing circuitry 111 determines whether or not the medication effect information has been calculated for all the dates included in the period of administration of the medicines “AAA Tablet” and “BBB Tablet” identified by the display instruction (step SB5). Since the medication effect information is not calculated for all the dates included in the period of administration of the medicines “AAA Tablet” and “BBB Tablet” (No in step SB5), the processing circuitry 111 shifts the processing to step SB2. The processing circuitry 111 calculates one-day medication effect information on the medicines “AAA Tablet” and “BBB Tablet” corresponding to Feb. 24, 2017 (Fri), which is the next day, from step SB2 through step SB4.

Similarly, the processing circuitry 111 calculates medication effect information on the medicine “AAA Tablet” during the period from Feb. 25, 2017 (Sat) to Feb. 28, 2017 (Tue), from step SB2 through step SB4.

Upon calculating the medication effect information during the period of administration (Yes in step SB5), the processing circuitry 111 calculates medication effect information after the period of administration, and determines the angle of the line to be displayed based on the calculated medication effect information (step SB6).

Upon calculating, for example, the medication effect information on the medicine “BBB Tablet” of Feb. 24, 2017 (Fri) (Yes in step SB5), the processing circuitry 111 calculates medication effect information of a day after the period of administration, i.e., of Feb. 25, 2017 (Sat), based on at least information on the in vivo kinetics contained in the package insert information. It is to be noted that, if the first analyzed information is merged with the package insert information and stored in the storage 113, such merged items of information may be used to calculate the medication effect information. The processing circuitry 111 determines, based on, for example, the medication effect information on the medicine “BBB Tablet”, calculated during the period from 0 o'clock to 24 o'clock of Feb. 25, 2017 (Sat), an angle of a line drawn from the value of the start point to the value of the final point during that period, with respect to the ruled line.

Upon calculating, for example, medication effect information on the medicine “AAA Tablet” (Yes in step SB5) of Feb. 28, 2017 (Tue), the processing circuitry 111 calculates medication effect information during a period after the period of administration, namely, during the period from Mar. 1, 2017 (Wed) to Mar. 4, 2017 (Sat), based on at least information on the in vivo kinetics contained in the package insert information. The processing circuitry 111 determines, based on, for example, the medication effect information on the medicine “AAA Tablet” calculated during the period from 0 o'clock to 24 o'clock of Mar. 1, 2017 (Wed) to Mar. 4, 2017 (Sat), the angle of the line drawn from the value of the start time to the value of the final point during that period, with respect to the ruled line.

When the angle of the line after the period of administration is determined, the processing circuitry 111 reads, for example, values representing the vital signs contained in the medical care information on a patient identified by the display instruction from the electronic medical record database 1131 (step SB7). Specifically, the processing circuitry 111 reads, for example, the body temperature, the pulse rate, the systolic blood pressure, the diastolic blood pressure, and the respiratory rate etc. of the patient identified by the display instruction, measured during the period from Feb. 23, 2017 (Thu) to Feb. 28, 2017 (Tue), from the electronic medical record database 1131.

The processing circuitry 111 generates display image data for displaying the progress of medical care, based on the acquired mode of administration, the angle of the line based on the medication effect information, and the read values representing the vital signs. Specifically, the processing circuitry 111 temporally associates the mode of administration, the angle of the line based on the medication effect information, and the values representing the read vital signs. Thereby, display image data which indicates, on a common time axis, information on the administration timing of a medicine and information on temporal changes of the medication effect is generated. Also, display image data which indicates, on a common time axis, information on the administration timing of a medicine, information on temporal changes of the medication effect, and information on temporal changes of the patient's condition is generated.

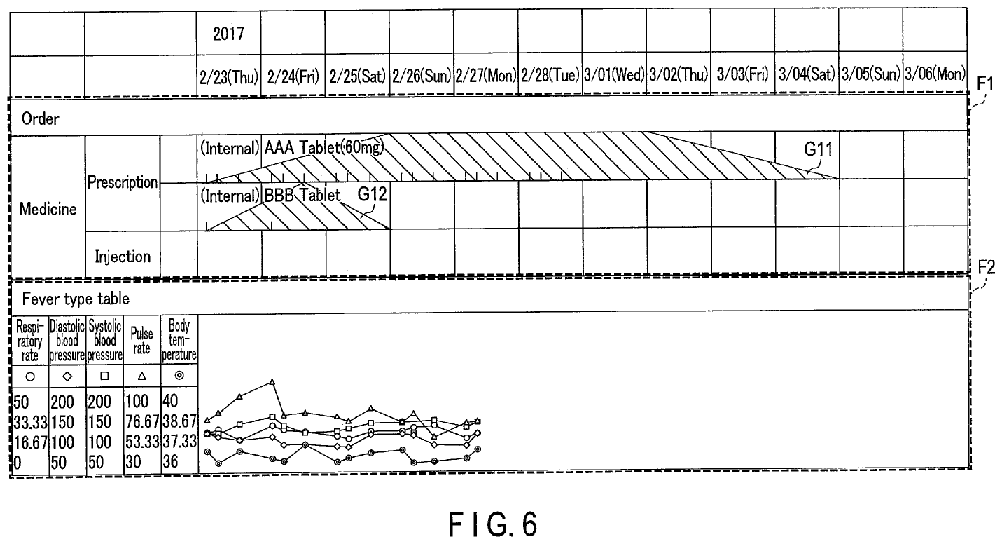

The generated display image data is output to the input/output apparatus 12. The display image data is displayed as a display image on a display device included in the input/output apparatus 12. FIG. 6 is a diagram illustrating an example in which the display image generated by the processing shown in FIG. 5 is displayed on the display device included in the electronic medical record system 1 shown in FIG. 1.

In FIG. 6, identifiers indicating the administration timings of the medicine, a region indicating temporal changes of the medication effect of the medicine, and a region indicating temporal changes of the condition of the patient into which the medicine has been administered are displayed on a common time axis. In the example of FIG. 6, the administration timing and temporal changes of the value representing the medication effect of the medicine on a specific patient during the period from Feb. 23, 2017 (Thu) to Mar. 6, 2017 (Mon) are displayed.

The display region F1 shown in FIG. 6 indicates the administration timings of the medicine “AAA Tablet” (60 mg per tablet), which is an internal drug, using vertical bars. Specifically, the display region F1 shown in FIG. 6 indicates that AAA Tablet should be administered in the morning, at noon, and in the evening of every day of administration during the period from Feb. 23, 2017 (Thu) to Feb. 28, 2017 (Tue).

The display region F1 shown in FIG. 6 indicates temporal changes of the value representing the medication effect of AAA Tablet in the form of the line graph G11. The vertical axis of the line graph G11 indicates the value representing the medication effect. The line graph G11 displayed in the display region F1 shown in FIG. 6 indicates, for example, that the ingredient of the medicine “AAA Tablet” is sufficiently absorbed into the patient, namely, that the medication effect is maximized (reaches 100%) during the period from Feb. 26, 2017 (Sun) to Mar. 1, 2017 (Wed). The line graph G11 displayed in the display region F1 shown in FIG. 6 indicates that the medication effect of AAA Tablet increases at a constant rate over the period from the timing of the first administration of “AAA Tablet” on Feb. 23, 2017 (Thu) to 24 o'clock of Feb. 25, 2017 (Sat). This allows healthcare professionals, etc. to perceive, for example, that the medication effect of “AAA Tablet” is approximately 50% at around 12 o'clock of Feb. 24, 2017 (Fri). The line graph G11 displayed in the display region F1 shown in FIG. 6 indicates that the medication effect of “AAA Tablet” decreases at a constant rate from 0 o'clock of Mar. 2, 2017 (Thu) to 24 o'clock of Mar. 4, 2017 (Sat). This allows healthcare professionals, etc. to perceive, for example, that the medication effect of “AAA Tablet” is reduced to approximately 0% at around 24 o'clock of Mar. 4, 2017 (Sat). In this manner, healthcare professionals, etc. are allowed to easily perceive temporal changes of the value representing the medication effect, and to evaluate the relationship between a medical care intervention and the patient's response to the intervention with higher precision, based on the acquired information on the medication effect. In addition, it becomes possible for healthcare professionals, etc. to perform their services appropriately, according to the perceived medication effect.

The display region F1 shown in FIG. 6 indicates the administration timings of the medicine “BBB Tablet”, which is an internal drug, using vertical bars. Specifically, the display region F1 shown in FIG. 6 indicates that the medicine “BBB Tablet” should be administered in the morning of each of Feb. 23, 2017 (Thu) and Feb. 24, 2017 (Fri).

The display region F1 shown in FIG. 6 indicates temporal changes of the value representing the medication effect of “BBB Tablet”, in the form of the line graph G12. The vertical axis of the line graph G12 indicates the value representing the medication effect. The line graph G12 displayed in the display region F1 shown in FIG. 6 indicates, for example, that the medication effect of “BBB Tablet” is maximized during a certain period of Feb. 24, 2017 (Fri). This allows healthcare professionals, etc. to perceive, for example, that the medication effect of “BBB. Tablet” is maximized at around 17 o'clock of Feb. 24, 2017 (Fri), making it possible to appropriately perform their services according to the timing when the medication effect is maximized.

In addition, the display region F2 shown in FIG. 6 indicates temporal changes of values representing vital signs of a specific patient, such as the body temperature, the pulse rate, the systolic blood pressure, the diastolic blood pressure, and the respiratory rate. The temporal changes of the values representing the vital signs are on the same time axis as the temporal changes of the value representing the medication effect included in the display region F1 shown in FIG. 6. It is thereby possible for healthcare professionals, etc. to observe the temporal changes of the value representing the medication effect, along with the condition of the patient.

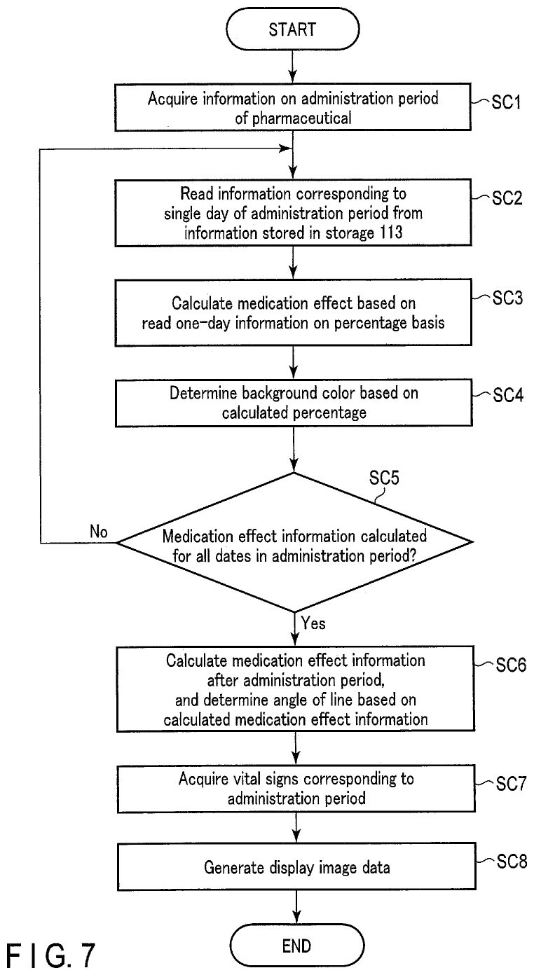

Next, a case will be described where the temporal changes of the value representing the medication effect are indicated on the display image as variations in color. FIG. 7 is a flowchart illustrating an operation of the processing circuitry 111 when the electronic medical record server 11 of the present embodiment generates display image data for a display image that indicates temporal changes of the value representing the medication effect as variations in color. In the description that follows, let us assume that multiple display colors are preset on a percentage basis as the colors to be displayed on the display image.

The operation from step SC1 to step SC3 shown in FIG. 7 is the same as the operation from step SB1 to step SB3 shown in FIG. 5.

The processing circuitry 111 determines the colors to be displayed on the screen, based on the calculated medication effect information, for each patient and medicine identified by the display instruction (step SC4). Specifically, the processing circuitry 111 determines, for example, display colors according to the medication effect information on each of the medicines “AAA Tablet” and “BBB Tablet” calculated during the period from 0 o'clock to 24 o'clock of Feb. 23, 2017 (Thu).

The operation from step SC5 to step SC8 shown in FIG. 7 is the same as the operation from step SB5 to step SB8 shown in FIG. 5.

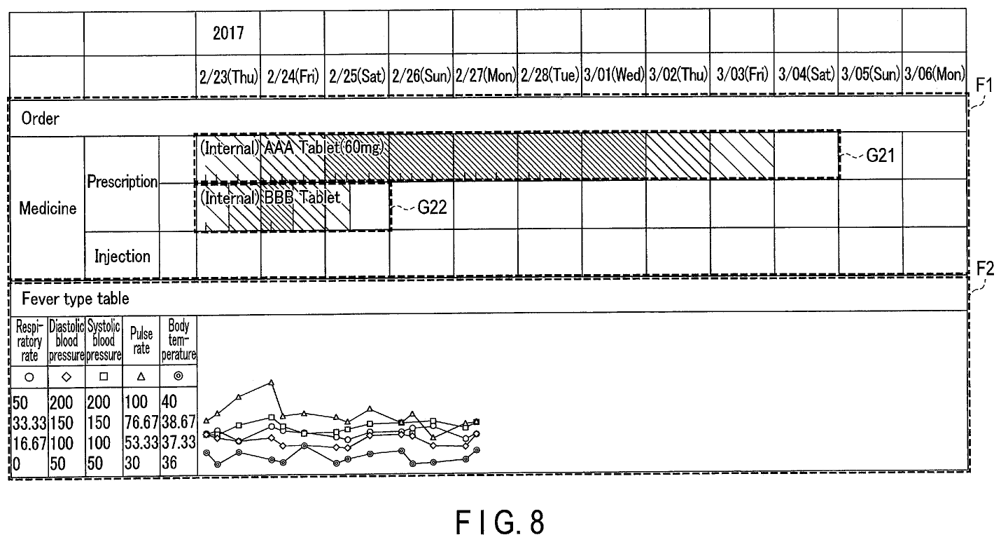

The display image data generated in step SB8 shown in FIG. 7 is output to the input/output apparatus 12. The display image data is displayed as a display image on a display device included in the input/output apparatus 12. FIG. 8 is a diagram illustrating an example in which a display image generated by the processing shown in FIG. 7 is displayed on the display device provided in the electronic medical record system 1 shown in FIG. 1.

In FIG. 8, identifiers indicating administration timings of a medicine, a region indicating temporal changes of the medication effect of the medicine, and a region indicating temporal changes of the condition of the patient into which the medicine has been administered are displayed on a common time axis. In the example of FIG. 8, temporal changes of the administration timing and the value representing the medication effect of the medicine relating to a specific patient during the period from Feb. 23, 2017 (Thu) to Mar. 6, 2017 (Mon) are displayed.

The display region F1 shown in FIG. 8 indicates administration timings of the medicine “AAA Tablet” (60 mg per tablet), which is an internal drug, using vertical bars. Specifically, the display region F1 shown in FIG. 8 indicates that “AAA Tablet” should be administered in the morning, at noon, and in the evening of every day of administration from Feb. 23, 2017 (Thu) to Feb. 28, 2017 (Tue).

The display region F1 shown in FIG. 8 indicates temporal changes of the value representing the medication effect of “AAA Tablet” as variations in color, in the form of the graph G21. The colors displayed in the respective regions of the graph G21 indicate the values representing the medication effects in a stepwise manner. That is, the colors displayed provide ranges in the value representing the medication effect. The graph G21 displayed in the display region F1 shown in FIG. 8 indicates, for example, temporal changes of the value representing the medication effect as three-step gradations in color. Specifically, the graph G21 displayed in the display region F1 shown in FIG. 8 indicates that the medication effect of “AAA Tablet” is equal to or greater than 0% and less than 50% during the period of Feb. 23, 2017 (Thu). The graph G21 displayed in the display region F1 shown in FIG. 8 indicates that the medication effect of “AAA Tablet” is, for example, equal to or greater than 50% and less than 100% during the period of Feb. 24, 2017 (Fri). The graph G21 displayed in the display region F1 shown in FIG. 8 indicates that the ingredient of the medicine “AAA Tablet” is sufficiently absorbed into the patient, namely, the medication effect is maximized during the period from Feb. 25, 2017 (Sat) to Mar. 1, 2017 (Wed). The graph G21 displayed in the display region F1 shown in FIG. 8 indicates that the medication effect of “AAA Tablet” is, for example, equal to or greater than 50% and less than 100% during the period of Mar. 2, 2017 (Thu). The graph G21 displayed in the display region F1 shown in FIG. 8 indicates that the medication effect of “AAA Tablet” equal to or greater than 0% and less than 50% during the period of Mar. 3, 2017 (Fri). This allows healthcare professionals, etc. to perceive the medication effect by color in a stepwise and intuitive manner, and to evaluate the relationship between a medical care intervention and the patient's response to the intervention based on the perceived medication effect. In addition, it becomes possible for healthcare professionals, etc. to perform their services appropriately, according to the perceived medication effect.

The display region F1 shown in FIG. 8 indicates the administration timings of the medicine “BBB Tablet”, which is an internal drug, using vertical bars. Specifically, the display region F1 shown in FIG. 8 indicates that the medicine “BBB Tablet” should be administered in the morning of each of Feb. 23, 2017 (Thu) and Feb. 24, 2017 (Fri).

The display region F1 shown in FIG. 8 indicates temporal changes of the value representing the medication effect of “BBB Tablet” as variations in color, in the form of the graph G22. The colors displayed in the respective regions of the graph G22 indicate the values representing the medication effects in a stepwise manner. The graph G22 displayed in the display region F1 shown in FIG. 8 indicates that the medication effect of “BBB Tablet” is maximized in the morning hours of Feb. 24, 2017 (Fri). This allows healthcare professionals, etc., to perceive, for example, that the medication effect of “BBB Tablet” is maximized in the morning hours of Feb. 24, 2017 (Fri), making it possible to appropriately perform their services according to the timing when the medication effect is maximized.

In addition, the display region F2 shown in FIG. 8 indicates temporal changes of the values representing vital signs of a specific patient, such as the body temperature, the pulse rate, the systolic blood pressure, the diastolic blood pressure, and the respiratory rate. The temporal changes of the values representing the vital signs are on the same time axis as the temporal changes of the value representing the medication effect included in the display region F1 shown in FIG. 8. It is thereby possible for healthcare professionals, etc. to observe the temporal changes of the value representing the medication effect, along with the condition of the patient.

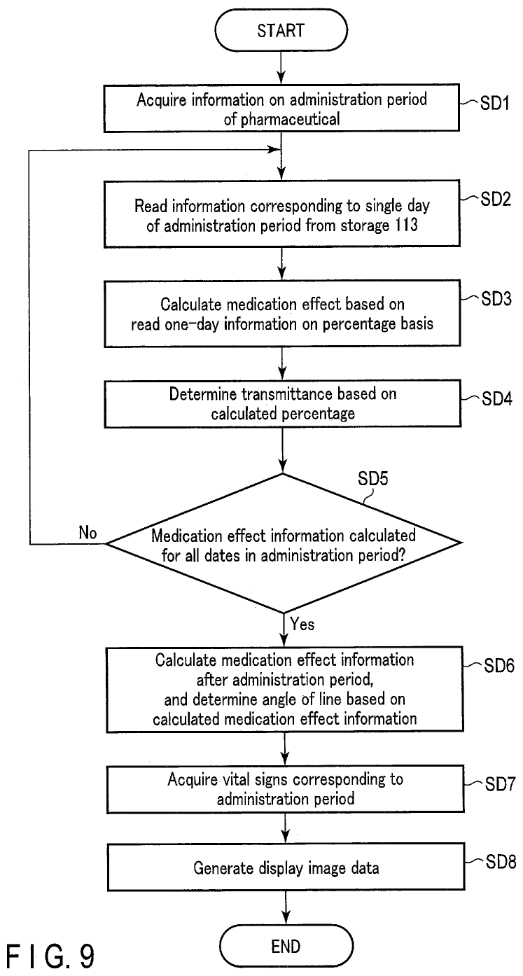

Finally, a case will be described where the temporal changes of the value representing the medication effect are indicated in the display image as variations in color transmittance. FIG. 9 is a flowchart illustrating an operation of the processing circuitry 111 when the electronic medical record server 11 of the present embodiment generates display image data for a display image that indicates temporal changes of the value representing the medication effect by color transmittance. In the description that follows, let us assume that multiple color transmittances are preset on a percentage basis, as the transmittances of the colors to be displayed on the display image.

The operation from step SD1 to step SD3 shown in FIG. 9 is the same as the operation from step SB1 to step SB3 shown in FIG. 5.

The processing circuitry 111 determines the color transmittances to be displayed on the screen based on the calculated medication effect information, for each patient and each medicine identified by the display instruction (step SD4). Specifically, the processing circuitry 111 determines, for example, the color transmittances according to the medication effect information on each of the medicines “AAA Tablet” and “BBB Tablet” calculated during the period from 0 o'clock to 24 o'clock of Feb. 23, 2017 (Thu).

The operation from step SD5 to step SD8 shown in FIG. 9 is the same as the operation from step SB5 to step SB8 shown in FIG. 5.

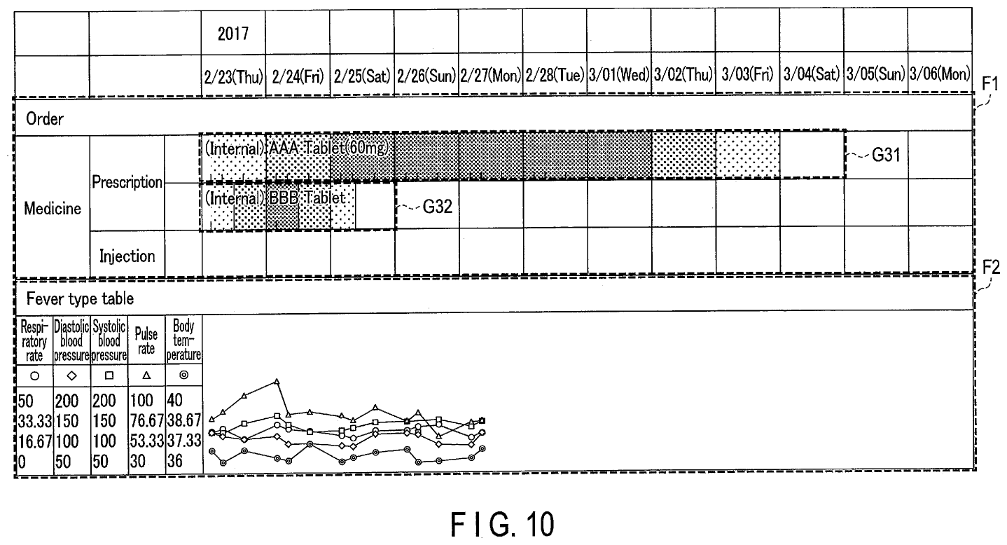

The display image data generated in step SD8 shown in FIG. 9 is output to the input/output apparatus 12. The display image data is displayed as a display image on a display device included in the input/output apparatus 12. FIG. 10 is a diagram illustrating an example in which a display image generated by the processing shown in FIG. 9 is displayed on the display device provided in the electronic medical record system 1 shown in FIG. 1.

In FIG. 10, identifiers indicating administration timings of a medicine, a region indicating temporal changes of the medication effect of the medicine, and a region indicating temporal changes of the condition of the patient into which the medicine has been administered are displayed on a common time axis. In the example of FIG. 10, the administration timing of the medicine and temporal changes of the value representing the medication effect relating to a specific patient during the period from Feb. 23, 2017 (Thu) to Mar. 6, 2017 (Mon) are displayed.

The display region F1 shown in FIG. 10 indicates the administration timings of the medicine “AAA Tablet” (60 mg per tablet), which is an internal drug, by vertical bars. Specifically, the display region F1 shown in FIG. 10 indicates that “AAA Tablet” should be administered in the morning, at noon, and in the evening of every day of administration from Feb. 23, 2017 (Thu) to Feb. 28, 2017 (Tue).

The display region F1 shown in FIG. 10 indicates temporal changes of the value representing the medication effect of “AAA Tablet” as variations in color transmittance, in the form of the graph G31. The color transmittances displayed in the respective regions of the graph G31 indicate the values representing the medication effects in a stepwise manner. That is, the color transmittances displayed provide ranges in the value representing the medication effects. The graph G31 displayed in the display region F1 shown in FIG. 10 indicates, for example, temporal changes of the value representing the medication effect as three-level color transmittances. Specifically, the graph G31 displayed in the display region F1 shown in FIG. 10 indicates that the medication effect of “AAA Tablet” during the period of Feb. 23, 2017 (Thu) is equal to or greater than 0% and less than 50%. The graph G31 displayed in the display region F1 shown in FIG. 10 further indicates that the medication effect of “AAA Tablet” is, for example, equal to or greater than 50% and less than 100% during the period of Feb. 24, 2017 (Fri). The graph G31 displayed in the display region F1 shown in FIG. 10 indicates that the ingredient of the medicine “AAA Tablet” is sufficiently absorbed into the patient, namely, the medication effect is maximized during the period from Feb. 25, 2017 (Sat) to Mar. 1, 2017 (Wed). The graph G31 displayed in the display region F1 shown in FIG. 10 indicates, for example, that the medication effect of “AAA Tablet” is equal to or greater than 50% and less than 100% during the period of Mar. 2, 2017 (Thu). The graph G31 displayed in the display region F1 shown in FIG. 10 indicates that the medication effect of “AAA Tablet” is equal to or greater than 0% and less than 50% during the period of Mar. 3, 2017 (Fri). This allows healthcare professionals, etc. to perceive the medication effect by color transmittance in a stepwise and intuitive manner, and to evaluate the relationship between a medical care intervention and the patient's response to the intervention based on the perceived medication effect. In addition, it becomes possible for healthcare professionals, etc. to perform their services appropriately, according to the perceived medication effect.

The display region F1 shown in FIG. 10 indicates the administration timings of the medicine “BBB Tablet”, which is an internal drug, using vertical bars. Specifically, the display region F1 shown in FIG. 10 indicates that “BBB Tablet” should be administered in the morning of each of Feb. 23, 2017 (Thu) and Feb. 24, 2017 (Fri).

The display region F1 shown in FIG. 10 indicates temporal changes of the value representing the medication effect of “BBB Tablet” as variations in color transmittance, in the form of the graph G32. The color transmittances displayed in the respective regions of the graph G32 indicate the values representing the medication effects in a stepwise manner. The graph G32 displayed in the display region F1 shown in FIG. 10 indicates that the medication effect of “BBB Tablet” is maximized in the morning hours of Feb. 24, 2017 (Fri). This allows healthcare professionals, etc. to perceive, for example, that the medication effect of “BBB Tablet” is maximized in the morning hours of Feb. 24, 2017 (Fri), making it possible to appropriately perform their services according to the timing when the medication effect is maximized.

In addition, the display region F2 shown in FIG. 10 indicates temporal changes of the values representing vital signs of a specific patient, such as the body temperature, the pulse rate, the systolic blood pressure, the diastolic blood pressure, and the respiratory rate. The temporal changes of the values representing the vital signs are on the same time axis as the temporal changes of the value representing the medication effect included in the display region F1 shown in FIG. 10. It is thereby possible for healthcare professionals, etc. to observe the temporal changes of the value representing the medication effect, along with the condition of the patient.

According to the present embodiment, the processing circuitry 111 acquires, through the information acquisition function 1111, information on the administration timing of the medicine to the patient, and information on temporal changes of the medication effect of the medicine. In addition, the processing circuitry 111 generates, through the display image data generation function 1112, image data representing information on the administration timing and information on temporal changes of the medication effect on a common time axis. It is thereby possible for healthcare professionals, etc. to perceive temporal changes of the medication effect of the medicine.

According to the present embodiment, the processing circuitry 111 acquires, through the information acquisition function 1111, information on temporal changes of the condition of the patient into which the medicine has been administered. The processing circuitry 111 indicates, through the display image data generation function 1112, the information on the temporal changes of the patient's condition on a common time axis in the image data. It is thereby possible for healthcare professionals, etc. to perceive temporal changes of the medication effect of the medicine, in view of the patient's condition.

According to the hospital information system according to the present embodiment, since the relationship between the administration of the medicine and its medication effect can be displayed more accurately on a medicine-by-medicine basis or on a patient-by-patient basis, it becomes possible for healthcare professionals, etc. to evaluate the relationship between a medical care intervention and a response to the intervention with higher precision.

(Modification)

In the above-described embodiment, a case has been described, as an example, where the electronic medical records stored in the electronic medical record database 1131 and the medical care big data stored in the data warehouse 4 have previously been analyzed. That is, the hospital information system according to the present embodiment acquires first analyzed information, which is a result of analysis of the electronic medical records, from the electronic medical record database 1131. In addition, the hospital information system acquires second analyzed information, which is a result of analysis of the medical care big data, from the data warehouse 4. In the modification, a case will be described where the hospital information system acquires pre-analysis medical care information from the electronic medical record database 1131 and/or the data warehouse 4, and analyzes the acquired medical care information.

The functional configuration of the hospital information system according to the modification is the same as the functional configuration of the hospital information system according to the embodiment shown in FIG. 1.

The information acquisition function 1111 equipped in the processing circuitry 111 of the electronic medical record server 11 according to the modification includes a function of analyzing acquired information, in addition to the functions equipped in the information acquisition function 1111. That is, the processing circuitry 111 is equipped with a function as an example of an analysis unit. Specifically, when the information acquisition function 1111 is implemented, the processing circuitry 111 acquires information contained in pre-analysis electronic medical records from the electronic medical record database 1131. The processing circuitry 111 analyzes the acquired information through the use of a predetermined data mining technique, such as machine learning and statistical analysis, and acquires first analyzed information. Also, the processing circuitry 111 acquires medical care big data from the data warehouse 4. The processing circuitry 111 analyzes the acquired medical care big data through the use of a predetermined data mining technique, such as machine learning and statistical analysis, and acquires second analyzed information.

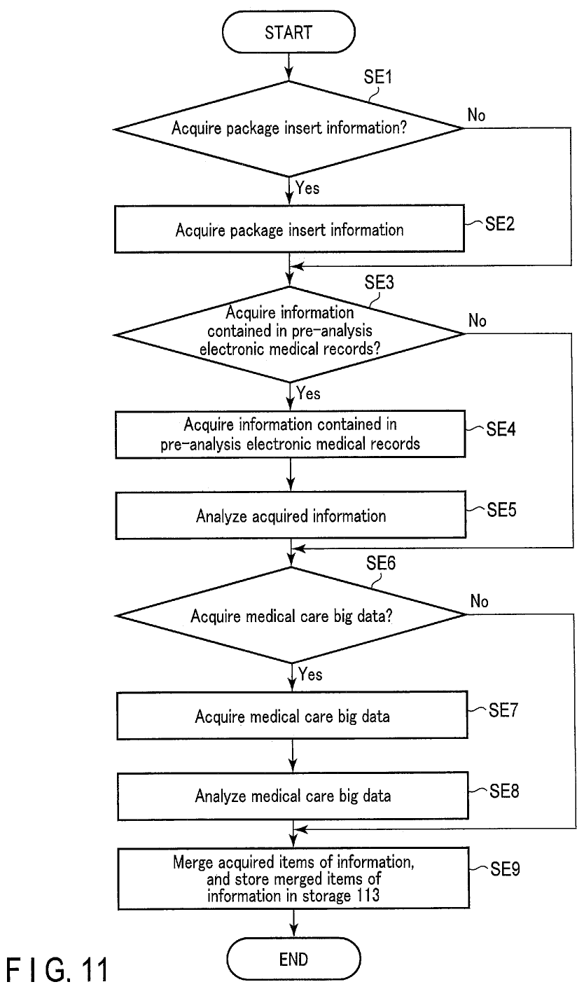

Next, an operation of the electronic medical record server 11 according to the modification will be described, in which various types of not-yet-analyzed medical care information is analyzed to generate display image data for displaying the progress of medical care, and the result of the analysis is acquired as first analyzed information. FIG. 11 is a flowchart illustrating the operation of the processing circuitry 111 when the electronic medical record server 11 according to the modification analyzes the various types of not-yet-analyzed medical care information, and acquires the results of the analysis as first analyzed information. In the description that follows, let us assume that options are preset as to which information is to be acquired, of the package insert information stored in the package insert information management server 3, the information contained in the pre-analysis electronic medical records stored in the electronic medical record database 1131, and the medical care big data stored in the data warehouse 4. Let us assume that information on the options is stored in, for example, the storage 113. Let us also assume that the preset information as the target to be acquired, is at least one of the package insert information, the information contained in the pre-analysis electronic medical records, and the medical care big data. The analysis of the medical care big data and the information contained in the pre-analysis electronic medical records may be performed by the processing circuitry 211 included in the diagnostic server 21 of the diagnostic system 2.

The processing circuitry 111 implements the information acquisition function 1111 when, for example, a preset batch process is started. Through the implementation of the information acquisition function 1111, the processing circuitry 111 refers to the information on the options stored in the storage 113, and determines whether the package insert information is an object to be acquired (step SE1). Upon determining that the package insert information is the object to be acquired (Yes in step SE1), the processing circuitry 111 acquires package insert information from the package insert information management server 3 via the communication interface 112 (step SE2).

Next, the processing circuitry 111 refers to information on the options stored in the storage 113, and determines whether or not the information contained in the pre-analysis electronic medical records is an object to be acquired (step SE3). Upon determining that the information contained in the pre-analysis electronic medical records is the object to be acquired (Yes in step SE3), the processing circuitry 111 acquires information contained in the pre-analysis electronic medical records from the electronic medical record database 1131 (step SE4).

The processing circuitry 111 analyzes the acquired information (step SE5). Specifically, the processing circuitry 111 takes the patient information, medical care information, etc. contained in the acquired information as input, and analyzes the medication effect on a patient-by-patient and on a medicine-by-medicine basis, namely, the degree to which the drug exerts effects on the patient into which the drug has been administered, through a predetermined data mining technique such as machine learning and statistical analysis. Examples of the machine learning include learning based on neural networks, decision tree analysis, learning based on support vector machines, etc. The machine learning may be either supervised or unsupervised. Examples of the statistical analysis include multiple regression analysis, principal components analysis, factor analysis, cluster analysis, etc. Thereby, first analyzed information is acquired. The actual records of administration of medicines to a patient contained in the medical care information may be directly used as information necessary for generating display image data, at the time of checking of the progress of the medical care to the same patient.

Next, the processing circuitry 111 refers to information on the options stored in the storage 113, and determines whether or not the medical care big data is an object to be acquired (step SE6). Upon determining that the medical care big data is the object to be acquired (Yes in step SE6), the processing circuitry 111 acquires medical care big data from the data warehouse 4 (step SE7).

The processing circuitry 111 analyzes the acquired medical care big data (step SE8). Specifically, the processing circuitry 111 analyzes patient information, medical care information, etc. contained in the medical care big data, through the use of a predetermined data mining technique such as machine learning and statistical analysis. Thereby, second analyzed information is acquired.

Finally, of the package insert information, the first analyzed information, and the second analyzed information, the processing circuitry 111 merges the package insert information, the first analyzed information, and the second analyzed information that have actually been acquired, and stores the merged items of information in the storage 113 (step SE9).

According to the modification, the processing circuitry 111 provided by the electronic medical record server 11 analyzes the information contained in the pre-analysis electronic medical records acquired from the electronic medical record database 1131, through the use of a predetermined data mining technique, and acquires first analyzed information. Also, the processing circuitry 111 analyzes the medical care big data acquired from the data warehouse 4 through the use of a predetermined data mining technique, and acquires second analyzed information. It is thereby possible to directly utilize the information accumulated in the electronic medical record database, and the medical care big data accumulated in the data warehouse 4.

### OTHER EMBODIMENTS

In the hospital information system according to the above-described embodiment, the display image data is generated in both the electronic medical record system and the diagnostic system; however, the present embodiment is not limited thereto. That is, at least one of the electronic medical record system and the diagnostic system may be configured to generate display image data.

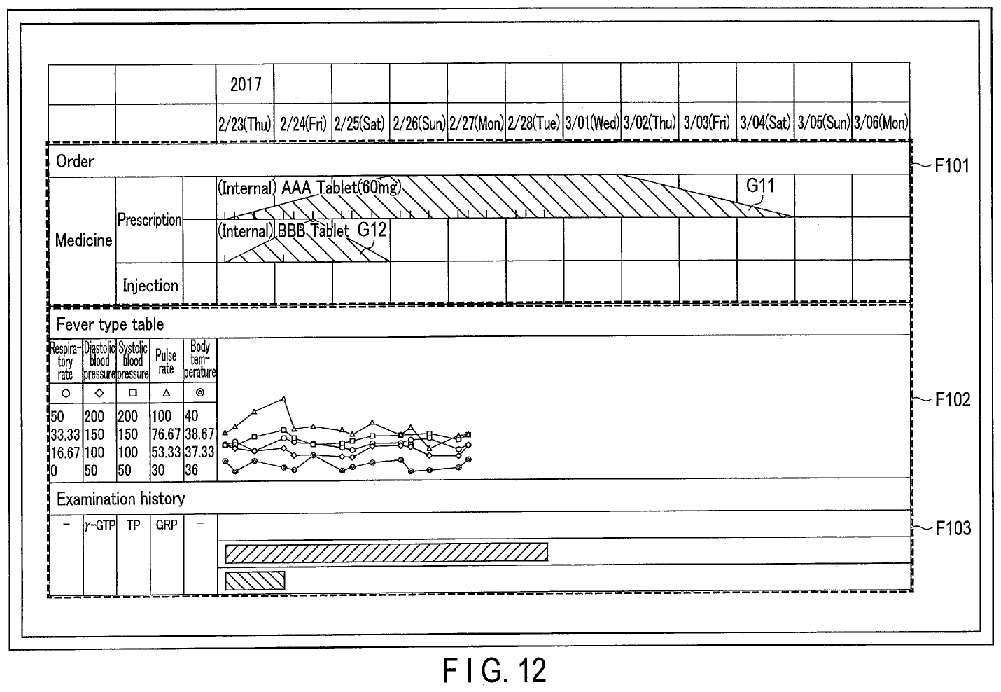

In the above-described embodiment, the period of time over which a medicine exerts effects is displayed in the display format shown in FIGS. 6, 8 and 10; however, the display format is not limited thereto. FIG. 12 is a diagram illustrating a first display example of a display image displayed on a display device included in an input/output apparatus of a diagnostic system according to another embodiment. FIG. 12 shows a display region F103 indicating an examination history of a patient into which a medicine has been administered, in addition to a display region F101 corresponding to the display region F1 shown in FIG. 6 and a display region 102 corresponding to the display region F2 shown in FIG. 6. The display region F103 is displayed along the timeline common to the display region F101 and the display region F102.

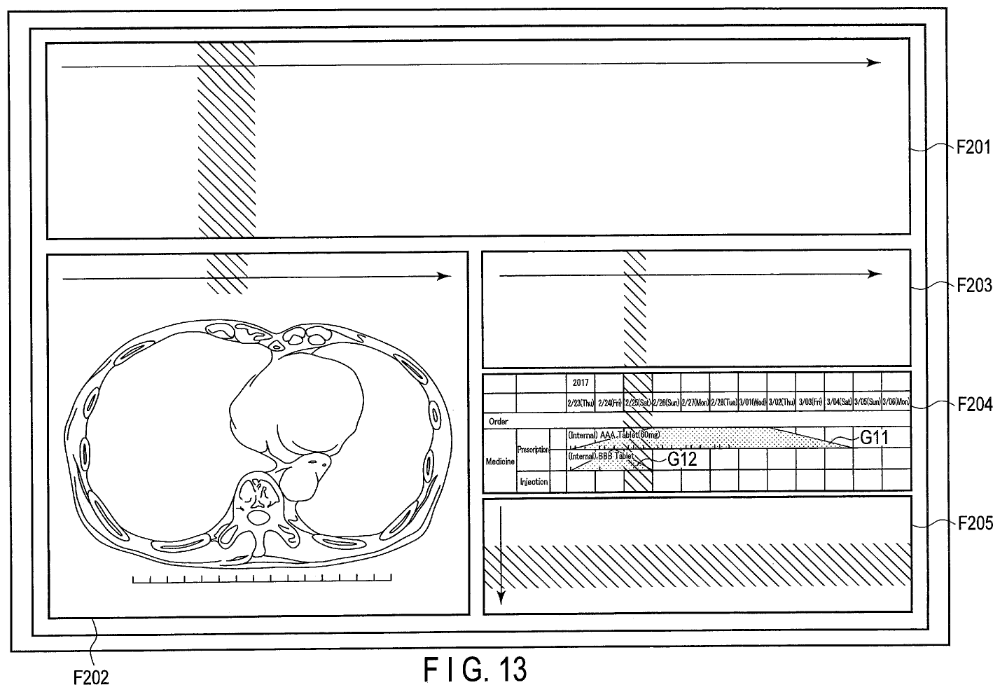

FIG. 13 is a diagram illustrating a second display example of a display image displayed on a display device included in an input/output apparatus of the diagnostic system according to another embodiment. FIG. 13 shows a display region F201 indicating an overall schedule of events, etc. related to medical care, a display region F202 indicating a medical image acquired from a patient, a display region F203 indicating various types of examination results of the patient, a display region F204 indicating medication effect information, and a display region F205 indicating order information, etc. In these display regions, a specific common time slot is associated and displayed in a display format such as highlighting.

With the display format shown in FIG. 12 or 13, it is possible for healthcare professionals, etc. to perceive the patient's condition in more detail, and to evaluate the relationship between administration of a medicine and its medication effect.

The term “processor” used in the above description refers to, for example, circuitry such as a central processing unit (CPU), a graphics processing unit (GPU), an application specific integrated circuit (ASIC), a programmable logic device such as a simple programmable logic device (SPLD), a complex programmable logic device (CPLD), or a field programmable gate array (FPGA), etc. The processor reads and executes programs stored in memory circuitry, thereby implementing the functions. The processors described in connection with the above embodiments are not limited to single-circuit processors; a plurality of independent processors may be integrated into a single processor that implements such functions. Furthermore, multiple structural components in FIGS. 1, 2, and 3 may be integrated into a single processor that implements such functions.

While some embodiments have been described, the embodiments have been presented as examples, and are not intended to limit the scope of the invention. These embodiments can be implemented in various other forms, and various omissions, replacements, and changes can be made without departing from the spirit of the invention. The embodiments and their modifications are included in the scope and spirit of the invention and are included in the scope of the claimed inventions and their equivalents.

What is claimed is:

1. A hospital information system comprising:
processing circuitry configured to:
acquire information on an administration timing of a medicine to a patient and information on temporal changes of a medication effect of the medicine; and
generate image data indicating the information on the administration timing and the information on temporal changes of the medication effect on a common time axis.

2. The hospital information system according to claim 1, wherein the processing circuitry
acquires information on temporal changes of a condition of the patient into which the medicine has been administered, and
indicates the information on the temporal changes of the condition of the patient on the common time axis in the image data.

3. The hospital information system according to claim 1, wherein the processing circuitry
indicates the information on the administration timing and the information on the temporal changes of the medication effect on an identical graph.

4. The hospital information system according to claim 2, wherein the processing circuitry
indicates the information on the administration timing, the information on the temporal changes of the medication effect, and the information on the temporal changes of the condition of the patient on an identical graph.

5. The hospital information system according to claim 1, wherein the processing circuitry
indicates the information on the temporal changes of the medication effect in a line graph.

6. The hospital information system according to claim 1, wherein the processing circuitry
indicates the information on the temporal changes of the medication effect by grayscale.

7. The hospital information system according to claim 1, wherein the processing circuitry
indicates the information on the temporal changes of the medication effect by transmittance.

8. The hospital information system according to claim 1, wherein the processing circuitry
analyzes patient information on the patient and medical care information, and
outputs the information on temporal changes of the medication effect of the medicine.

9. The hospital information system according to claim 8, wherein personally identifiable information is deleted from the patient information.

10. The hospital information system according to claim 1, wherein the processing circuitry
acquires the information on the temporal changes of the medication effect of the medicine, based on information included in package insert information included in a pharmaceutical medicine, and indicating a relationship between a blood concentration in the patient into which the pharmaceutical medicine has been administered and the time elapsed since the administration.

11. The hospital information system according to claim 1, wherein the information on the temporal changes of the condition of the patient includes values representing vital signs acquired through measurement of the patient.

12. The hospital information system according to claim 1, wherein the processing circuitry
indicates the information on the temporal changes of the medication effect on a percentage basis, assuming that a predetermined value is 100.

13. The hospital information system according to claim 1, wherein the processing circuitry
displays an image based on image data.

14. An image data generation method comprising:
acquiring information on an administration timing of a medicine to a patient and information on temporal changes of a medication effect of the medicine; and
generating image data indicating the information on the administration timing and the information on temporal changes of the medication effect on a common time axis.
